(19) United States
(12) Patent Application Publication
(10) Pub. No.: US 2021/0290874 A1
(43) Pub. Date: Sep. 23, 2021

(54) MEDICAL VENTILATOR WITH AIR INLETS THAT ARE PROTECTED BY FILTERS ARRANGED IN A DETACHABLE COMMON FILTRATION CARTRIDGE

(71) Applicant: Air Liquide Medical Systems, Antony (FR)

(72) Inventors: Romain DAVOINE, Grenoble (FR); Pauline GIARD, Saint Germain le Vasson (FR); Nicolas LABETTEUR, Bretigny sur Orge (FR)

(21) Appl. No.: 17/204,422

(22) Filed: Mar. 17, 2021

(30) Foreign Application Priority Data
Mar. 18, 2020 (FR) 2002670

(51) Int. Cl. A61M 16/00; A61M 16/10

(52) U.S. Cl. CPC A61M 16/0066; A61M 16/105; A61M 2205/8206; A61M 2205/3606; A61M 2205/502; A61M 2205/12

(57) ABSTRACT

The invention relates to a medical ventilator comprising a motorized micro-blower and electronic components; a patient air circuit with a patient air inlet to a suction inlet of the micro-blower in order to supply the micro-blower with air; and a cooling air circuit comprising a cooling air inlet in order to convey cooling air which, by thermal exchange, is intended to cool at least some of the electronic components. The patient air inlet is equipped with a first filter, and the cooling air inlet is equipped with a second filter. The filters are arranged in a common filtration cartridge, which is fastened detachably to the ventilator at the region of the air inlets.

### CROSS REFERENCE TO RELATED APPLICATIONS

This application claims the benefit of priority under 35 U.S.C. § 119 (a) and (b) to French Patent Application No. 2002679, filed Mar. 18, 2020, the entire contents of which are incorporated herein by reference.

### BACKGROUND

The invention relates to a medical ventilator in which the patient air inlet of the patient air circuit and the cooling air inlet of the cooling air circuit are equipped with filters that are carried by a common filtration cartridge fastened detachably, i.e. removably, so as to be able to be mounted and unmounted easily from outside the ventilator, that is to say without the need to open the shell of the ventilator or to access the interior of the latter, and also a filtration cartridge of this kind.

In a hospital or in the field, the treatment of a patient suffering from respiratory insufficiency, whether in an emergency or not, can be carried out using a respiratory assistance apparatus or medical ventilator supplying air, possibly with addition of oxygen, that is to say oxygen-enriched air.

The medical ventilator has to permit ventilation of a patient in “normal” environments, for example in a building, in a town, on a country road or the like, but also in “difficult” environments, in particular in environments contaminated by pollutants such as dust, pollen, bacteria or the like, or in environments where it may be hot, for example in high summer or in desert regions or tropical regions.

In order to ensure a level of safety and protection of the patient during treatment of the latter by administration of air or of oxygen-enriched air, it is necessary to be able to filter the ambient air entering the ventilator in order to free it of its atmospheric pollutants before it is administered to the patient.

In addition, a medical ventilator is caused to heat up on account of its electromechanical composition, especially on account of the presence of electronic components such as a digital screen, a micro-blower with electric motor, one or more electronic boards with microprocessor(s), one or more internal batteries, etc., and this heating may be accentuated in hot environments.

For this reason, it is necessary to cool the ventilator, and this is commonly done by flushing the interior of the ventilator with a flow of cooling air that serves to evacuate the heat to the outside, However, in order to avoid contamination of the interior of the medical ventilator by atmospheric pollutants, it is again necessary here to filter the cooling air serving to cool the medical ventilator.

To do this, the medical ventilators are often equipped with filters arranged on the respiratory gas circuit and on the cooling air circuit of these ventilators. However, replacing these filters is complicated and time-consuming, because they are arranged in the shell of the ventilators, which means that the ventilator has to be dismantled in order to allow a maintenance operator to access the filters and change them when they are saturated.

DE-A-10 2014 009 895 proposes a respiratory apparatus comprising two separate gas inlets, each of them provided with a filter. It is not clearly explained how to proceed to replace the filters. However, in view of the architecture of this apparatus, each filter has to be withdrawn and changed independently of the other.

These unmounting and remounting operations are all the more time-consuming because the replacements are frequent. Therefore, depending on the environments in which the ventilators are used, the filters sometimes have to be replaced several times a year.

The problem is to make available a medical ventilator of simplified architecture, in particular configured to permit easy and quick replacement of the air filters protecting the respiratory gas circuit and the cooling air circuit of the medical ventilator.

### SUMMARY

The solution provided by the invention concerns a medical ventilator comprising a motorized micro-blower and electronic components, and additionally comprising:
a patient air circuit fluidically connecting a patient air inlet to a suction inlet of the micro-blower in order to supply the micro-blower with air, and
a cooling air circuit comprising a cooling air inlet in order to convey cooling air which, by thermal exchange, is intended to cool at least some of the electronic components,

and in which the patient air inlet of the patient air circuit is equipped with a first filter, and the cooling air inlet of the cooling air circuit is equipped with a second filter.

According to the invention, the first and second filters are carried by a detachable (i.e. removable) common filtration cartridge.

Depending on the embodiment in question, the medical ventilator of the invention can comprise one or more of the following features:
the detachable common filtration cartridge is configured to be able to be mounted and unmounted from outside the medical ventilator, especially for maintenance or replacement of the filters.
said first and second filters are rigidly connected to the filtration cartridge so as to be able to be simultaneously withdrawn from or inserted, i.e. positioned, in the medical ventilator upon withdrawal or insertion, respectively, of the common filtration cartridge from or into the inside of the medical ventilator.
the patient air circuit and the cooling air circuit comprise gas passages or conduits or similar.
it comprises a rigid outer shell.
the shell of the medical ventilator is made of polymer.
the motorized micro-blower and the electronic components are arranged in the shell of the ventilator.
the common filtration cartridge comprising the first and second filters is fastened detachably to the ventilator.
the common filtration cartridge comprising the first and second filters is fastened detachably to the shell of the ventilator.
the filtration cartridge is configured to fasten detachably to the ventilator at the region of the patient air inlet of the patient air circuit and the cooling air inlet of the cooling air circuit, preferably to the shell of the ventilator.
the patient air inlet of the patient air circuit and the cooling air inlet of the cooling air circuit are situated in at least one inlet compartment open towards the outside, that is to say having an opening communicating to the outside of the ventilator.
it comprises means for circulation of the cooling air, for example a cooling fan and/or the natural convection and/or deflectors for orienting the cooling air.
the inlet compartment is formed in the shell of the medical ventilator.
the inlet compartment is configured to accommodate the filtration cartridge comprising the first and second filters.
the inlet compartment comprises an internal separation wall defining a first chamber and a second chamber.
the first filter is accommodated in the first chamber and the second filter is accommodated in the second chamber when the filtration cartridge is inserted into the inlet compartment.
the filtration cartridge comprises a space separating the first filter and second filter, the internal separation wall of the inlet compartment of the ventilator being accommodated in the space of the filtration cartridge when said filtration cartridge is inserted into the inlet compartment of the ventilator.
the filtration cartridge comprises a support frame made of polymer and carrying the first and second filters.
the filtration cartridge comprises fastening means for fastening it detachably to the ventilator, for example the fastening means comprise clips incorporated in the filtration cartridge, fastening screws or the like, or a threading.
the cooling air circuit is configured to convey cooling air in order, by (direct or indirect) thermal exchange, to cool one or more electronic components chosen from among an electronic board with microprocessor(s), a human-machine interface or HMI screen, an internal battery or a micro-blower motor.
the cooling air circuit is configured to carry out a gas flush, with a flow of cooling air, of one or more electronic components of the ventilator in such a way as to obtain a thermal exchange (i.e. a transfer of heat) between the flow of cooling air and the one or more electronic components that are to be cooled.
the micro-blower, also called turbine or compressor, comprises an electric motor.
the micro-blower comprises a protective housing protecting the rotor and the stator.
the micro-blower is accommodated in an internal casing arranged in the shell of the ventilator.
the electronic board with microprocessor(s) is configured to control the micro-blower.
the one or more internal batteries are rechargeable.
the one or more internal batteries supply electric current to at least the micro-blower, the electronic board and the HMI screen.
the first filter and the second filter are identical or different.
the first filter and the second filter are chosen to stop pollutants, i.e. particles, that have a size greater than or equal to 0.3 μm.
the first filter and the second filter are high-efficiency air filters, that is to say HEPA filters.
the micro-blower comprises a gas outlet fluidically supplying a circuit which delivers gas to the patient, in particular air or an air/O₂ mixture, and which leads to a gas outlet port.
the cooling air circuit comprises at least one hot air evacuation outlet permitting evacuation to the ambient atmosphere, the air having been heated by thermal exchange (i.e. exchange of heat) with the electronic components of the ventilator.

The invention also relates to a filtration cartridge for a medical ventilator according to the invention, characterized in that it comprises a support frame carrying the first and second filters, said first and second filters being spaced apart from each other by a space.

Depending on the embodiment in question, the filtration cartridge of the invention can comprise one or more of the following features:
the support frame is made of polymer, for example thermoplastic, especially ABS, ABS-PC or PLA.
the space has a slit shape, that is to say a space of elongate shape.
the support frame comprises an outer wall through which air passage orifices extend, for example circular orifices or similar, slits or the like.
the first filter has a size greater than that of the second filter, preferably being twice the size.
the first and second filters preferably have the shape of a parallelepiped, in particular a rectangular parallelepiped.
the support frame comprises fluidic sealing means, such as a lip, a gasket or the like.
the support frame comprises fastening means configured to permit fastening, i.e. detachable connection, of the filtration cartridge inside the medical ventilator, for example by interlocking, clipping, screwing or the like.
the first and second filters are HEPA filters.
the first and second filters are configured to stop pollutants, i.e. particles, that have a size greater than or equal to 0.3 μm.

### BRIEF DESCRIPTION OF THE DRAWINGS

The invention will now be better understood from the following detailed description, which is provided by way of a non-limiting illustration, with reference to the appended figures, in which:

FIG. 1 is a schematic representation of a medical ventilator equipped with a filtration cartridge according to the invention,

FIG. 2 is a front view of the filtration cartridge inserted into the medical ventilator from FIG. 1,

FIG. 3 is another view of the medical ventilator from FIG. 1,

FIG. 4 illustrates the mounting of the filtration cartridge in the medical ventilator from FIG. 1,

FIG. 5 is a schematic representation of the filtration cartridge and the internal gas circuits of the medical ventilator from FIG. 1,

FIG. 6 is a front view of the filtration cartridge of the medical ventilator from FIG. 5, and

FIG. 7 is a three-dimensional view of the filtration cartridge inserted into the medical ventilator from FIG. 1.

### DETAILED DESCRIPTION OF PREFERRED EMBODIMENTS

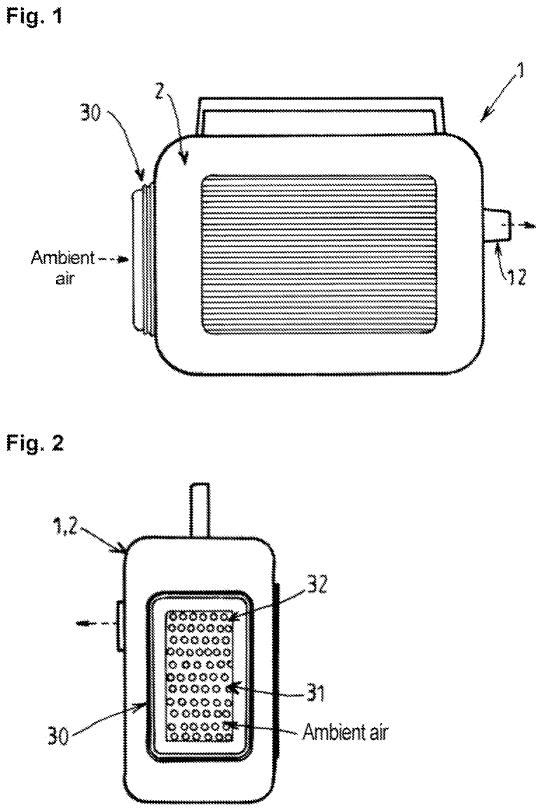

FIG. 1 is a schematic representation of a medical ventilator 1, for example an emergency ventilator, equipped with a filtration cartridge 30 comprising the first filter 31 and second filter 32 according to the invention. As is illustrated in FIG. 2, the filtration cartridge 30 is inserted into one of the lateral faces of the shell 2 of the ventilator 1,

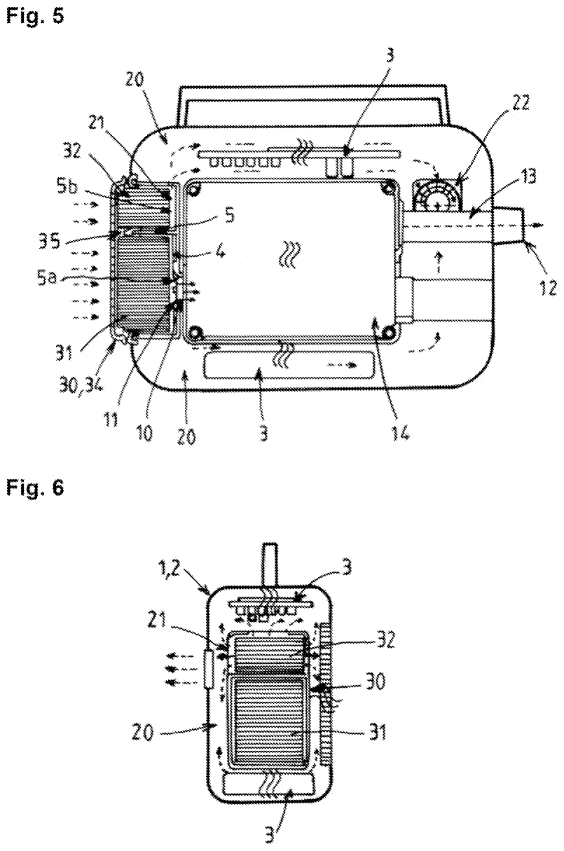

More precisely, and as shown schematically in FIG. 5 and FIG. 6, the first filter 31 is arranged at the patient air inlet 11 of the patient air circuit 10 of the ventilator 1, while the second filter 32 is arranged at the cooling air inlet 21 of the cooling air circuit 20 of the ventilator 1, each of them being fastened detachably, that is to say removably, so as to be able to be mounted and unmounted easily and quickly by an operator, directly from outside the ventilator 1, that is to say without the operator having to access the interior of the shell 2 of the medical ventilator 1.

In FIGS. 1 to 6, the flows of gas are indicated schematically by arrows in broken lines (- - - >).

The first filter 31 can be identical to the second filter 32 or, if appropriate, they can be different from each other, that is to say formed of different filtration media.

The ambient air may be naturally charged with atmospheric pollutants such as particles, dust, pollen, bacteria or the like, which it is necessary to hold back so as not to pollute the air circuits 10, 20 of the ventilator 1. Preferably, the filtration media of the first and second filters 31, 32 have an ability to retain atmospheric pollutants having dimensions of greater than 0.3 μm, for example high-efficiency particulate air (HEPA) filters. For example, the first and second filters 31, 32 can be formed of paper.

As is shown schematically in FIG. 5, the medical ventilator 1 comprises, arranged within its rigid outer shell 2, a micro-blower with an electric motor (not visible) and other electronic components 3 that need to be cooled during the operation of the medical ventilator 1, for example an electronic board with microprocessor, an HMI screen, an internal battery and/or the micro-blower motor.

To do this, a gas flush is carried out with cooling air supplied by the cooling air circuit 20 of the ventilator 1, which is configured to convey cooling air into direct or indirect contact with the electronic components 3 that are to be cooled, so as to effect a thermal exchange between the air and the electronic components 3 and thereby cool the latter by heat exchange.

The cooling air circuit 20 of the ventilator 1 comprises the cooling air inlet 21 at which the second filter 32 is inserted.

The circulation of cooling air is effected via a cooling fan 22 in FIG. 5 or, depending on the circumstances, by natural convection and/or via deflectors for orienting the flow or air.

Moreover, a patient air circuit 10 is also provided, which fluidically connects the patient aft inlet 11 to a suction inlet of the micro-blower in such way as to deliver air to the micro-blower. The air is aspirated by the micro-blower during the operation thereof. The air leaves via the gas outlet of the micro-blower and fluidically supplies a patient gas delivery circuit 13 which leads to a gas outlet port 12 arranged on the shell 2 of the ventilator 1. The gas delivered via the gas outlet port 12 of the ventilator 1 can be air or an air/O₂ mixture. It is then conveyed to the patient via a flexible conduit which is fluidically connected to the gas outlet port 12 via one of its ends and which, at its other end, comprises a patient respiration interface, for example a breathing mask, serving to distribute the gas to the patient.

In FIG. 5, the motorized micro-blower is arranged in a casing 14 through which a part of the patient air circuit 10 passes.

The motorized micro-blower and the other electronic components 3 requiring electric current to function, in particular an electronic board with microprocessor controlling the micro-blower, are supplied with electric current via one or more preferably rechargeable batteries.

Preferably, as is illustrated in FIG. 4 to FIG. 7, the first and second filters 31, 32 are carried by a common filtration cartridge 30 which is configured to fasten detachably to the ventilator 1 at the region of the patient air inlet 11 of the patient air circuit 10 and the cooling air inlet 21 of the cooling air circuit 20.

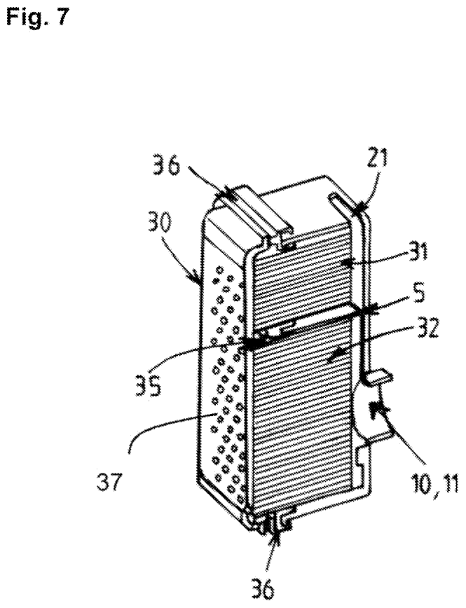

The filtration cartridge 30 can be in the form of a support frame 34, for example made of polymer, carrying the first and second filters 31, 32, said filters being separated from each other by a space 35, as is illustrated in FIG. 5 and FIG. 7. For example, the filtration cartridge 30 can be made of thermoplastic, especially of ABS, ABS-PC or PLA.

Furthermore, the patient air inlet 11 of the patient air circuit 10 and the cooling air inlet 21 of the cooling air circuit 20 are situated in an inlet compartment 4 open towards the outside, which is formed in the shell 2 of the ventilator 1. This inlet compartment 4 is configured, i.e. designed and dimensioned, to accommodate, in a removable manner, the filtration cartridge 30 equipped with the first and second filters 31, 32.

More precisely, the inlet compartment 4 of the ventilator 1 comprises an internal separation wall 5 defining a first chamber 5a and a second chamber 5b. The first filter 31 is accommodated in the first chamber 5a and the second filter 32 is accommodated in the second chamber 5b when the filtration cartridge 30 is inserted into the inlet compartment 4. Furthermore, the internal separation wall 5 of the inlet compartment 4 will for its part be accommodated in the space 35 of the filtration cartridge 30, as is shown in FIG. 5.

In the embodiment shown, the first and second chambers 5a, 5b have different sizes, since the first filter 31 is also of a larger size than the second filter 32, because the quantity of air that is to be supplied to the patient circuit 10 is greater than the quantity that is to be supplied to the cooling circuit 20.

For example, the total volume occupied by the two filters 31 32 can be of the order of 100×50×35 mm, with a distribution of ⅓ for the second filter 32 and of ⅔ for the first filter 31, that is to say the first filter 31 is twice the size of the second filter 32. They can have a shape that is approximately a rectangular parallelepiped.

The mounting or unmounting of the filtration cartridge 30 is illustrated in FIG. 4. As will be seen, the filtration cartridge 30 comprising the first and second filters 31, 32 is easily inserted or removed from outside the ventilator 1, without the shell 2 having to be opened.

The filtration cartridge 30 is fastened within the inlet compartment 4 of the ventilator 1 by fastening means, for example by interlocking, clipping or screwing. The leaktightness is provided by sealing means or a sealing device 36, such as a lip seal or the like, for example a sealing gasket made of thermoplastic, elastomer or silicone, visible in FIG. 7.

Furthermore, the support frame 34 of the filtration cartridge 30 comprises an outer wall 37 through which air passage orifices 38 extend, allowing the ambient air to pass through and then be filtered by the filters 31, 32. The outer wall 37 is directed towards the outside when the filtration cartridge 30 is inserted into the inlet compartment 4 of the ventilator 1. This outer wall 37 then forms a lid or roof that closes the opening of the inlet compartment 4 of the ventilator 1, as is illustrated in FIG. 5 and FIG. 6.

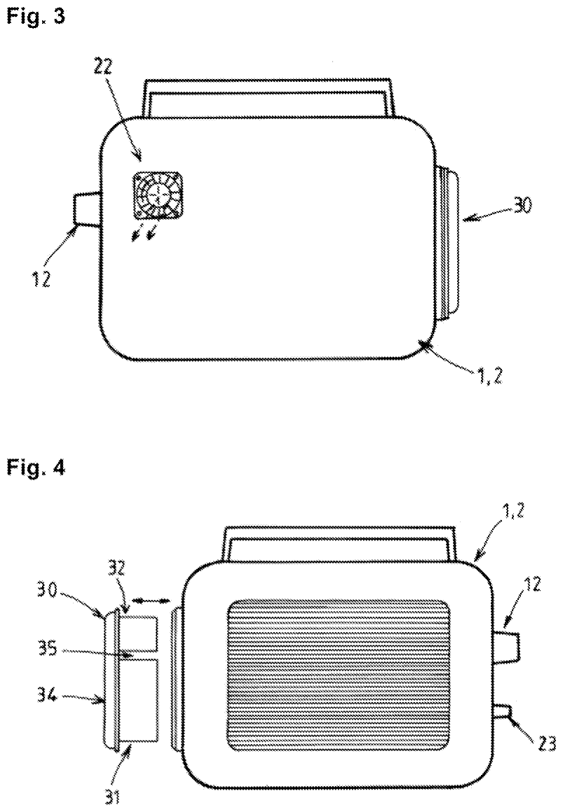

The cooling air, after being heated upon contact with the other electronic components 3 of the medical ventilator 1, can be evacuated to the outside, that is to say to the ambient atmosphere, via one or more openings arranged through the wall of the shell 2 of the medical ventilator 1, as is illustrated in FIG. 3, in particular via a cooling fan 22 illustrated in FIG. 5.

Furthermore, in order to be able to enrich the air aspirated by the micro-blower, an oxygen inlet 23, as can be seen in FIG. 3, is provided which can be fluidically connected to a source of gaseous oxygen, for example to a cylinder of compressed oxygen or to an oxygen duct that forms part of the network of gas ducts in a hospital building. The mixing of air and oxygen preferably takes place upstream from the micro-blower, for example in the casing 14 before entry into the micro-blower.

For its part, the micro-blower is a conventional micro-blower comprising an electric motor equipped with a rotary shaft or axle supporting a bladed wheel arranged in a wheel compartment of a volute. The wheel is movable in rotation in the wheel compartment by being driven by the electric motor, during its operation, in order to deliver a flow of respiratory gas (air or air/O₂) in the gas delivery circuit 13 terminated by the gas outlet port 12.

It will be noted that, according to a particular embodiment, the one or more filters could be filters of the NRBC type (nuclear, radiological, biological, chemical), permitting protection of the patient and of the machine in this environment.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A medical ventilator (1) comprising a motorized micro-blower and electronic components (3), and additionally comprising:
a patient air circuit (10) fluidically connecting a patient air inlet (11) to a suction inlet of the micro-blower in order to supply the micro-blower with air, and
a cooling air circuit (20) comprising a cooling air inlet (21) in order to convey cooling air which, by thermal exchange, is intended to cool at least some of the electronic components (3),
and in which the patient air inlet (11) of the patient air circuit (10) is equipped with a first filter (31), and the cooling air inlet (21) of the cooling air circuit (20) is equipped with a second filter (32),
characterized in that the first and second filters (31, 32) are carried by a detachable common filtration cartridge (30).

2. The ventilator according to claim 1, characterized in that the filtration cartridge (30) is configured to fasten detachably to the ventilator (1) at the region of the patient air inlet (11) of the patient air circuit (10) and the cooling air inlet (21) of the cooling air circuit (20).

3. The ventilator according to claim 1, characterized in that the ventilator (1) comprises a rigid shell, the filtration cartridge (30) being configured to fasten detachably to the rigid shell of the ventilator (1).

4. The ventilator according to claim 1, characterized in that the patient air inlet (11) of the patient air circuit (10) and the cooling air inlet (21) of the cooling air circuit (20) are situated in at least one inlet compartment (4) open towards the outside from the ventilator (1).

5. The ventilator according to claim 4, characterized in that the inlet compartment (4) is configured to accommodate the filtration cartridge (30) having the first and second filters (31, 32).

6. The ventilator according to claim 4, characterized in that the inlet compartment (4) comprises an internal separation wall (5) defining a first chamber (5a) and a second chamber (5b), the first filter (31) being accommodated in the first chamber (5a) and the second filter (32) being accommodated in the second chamber (5b) when the filtration cartridge (30) is inserted into the inlet compartment (4).

7. The ventilator according to claim 6, characterized in that the filtration cartridge (30) comprises a space (35) separating the first filter (31) and second filter (32), the internal separation wall (5) of the inlet compartment (4) of the ventilator (1) being accommodated in the space (35) of the filtration cartridge (30) when said filtration cartridge (30) is inserted into the inlet compartment (4) of the ventilator (1).

8. The ventilator according to claim 1, characterized in that the filtration cartridge (30) comprises a support frame (34) carrying the first and second filters (31, 32).

9. The ventilator according to claim 1, characterized in that the cooling air circuit (20) is configured to convey cooling air in order, by thermal exchange, to cool one or more electronic components (3) chosen from among an electronic board with microprocessor, an HMI screen, an internal battery or a micro-blower motor.

10. The ventilator according to claim 1, characterized in that the first and second filters (31, 32) are chosen to stop pollutants having a size of greater than or equal to 0.3 μm.

11. the ventilator according to claim 1, characterized in that the first and second filters (31, 32) are HEPA filters.

12. The ventilator according to claim 1, characterized in that the detachable common filtration cartridge (30) is configured to be mounted and unmounted from outside the ventilator (1).

13. A filtration cartridge (30) for a medical ventilator (1) according to claim 1, characterized in that the filtration cartridge (30) comprises a support frame (34) carrying the first and second filters (31, 32), said first and second filters (31, 32) being spaced apart from each other by a space (35).

14. The filtration cartridge according to claim 13, characterized in that the support frame (34) comprises:
an outer wall (37) through which air passage orifices (38) extend,
fluidic sealing means (36) and/or
fastening element configured to avow the filtration cartridge (30) to be fastened inside the medical ventilator (1).

15. The filtration cartridge (30) according to claim 13, characterized in that the first filter (31) has a size greater than that of the second filter (32).
